(19) United States
(12) Patent Application Publication
(10) Pub. No.: US 2018/0256102 A1
(43) Pub. Date: Sep. 13, 2018

(54) NEURAL ELECTRODE DEVICE COMPRISING NEURAL ELECTRODE ARRAY AND MICRO FLUIDIC CHANNEL FOR DELIVERING DRUG

(71) Applicant: DAEGU GYEONGBUK INSTITUTE OF SCIENCE & TECHNOLOGY, Daegu (KR)

(72) Inventors: So Hee KIM, Daegu (KR); Nam Sun CHOU, Seoul (KR); Yoo Na KANG, Daegu (KR); Won Ju CHUN, Daegu (KR)

(73) Assignee: DAEGU GYEONGBUK INSTITUTE OF SCIENCE & TECHNOLOGY, Daegu (KR)

(21) Appl. No.: 15/915,705

(22) Filed: Mar. 8, 2018

(30) Foreign Application Priority Data
Mar. 8, 2017 (KR) 10-2017-0029646

(51) Int. Cl. A61B 5/00; A61B 5/04

(52) U.S. Cl. CPC A61B 5/4839; A61B 5/0482; A61B 2562/0209; A61B 5/04001

(57) ABSTRACT

An exemplary neural electrode device as disclosed includes a storage in which fluid is stored; an electrode array which delivers the fluid to a subject, acquires bio-signals from the subject, or provides stimulus to the subject; an electronic circuit unit which allows the fluid to flow from the storage to the electrode array, processes the bio-signals acquired from the electrode array, or provides stimulus signals to the electrode array; and a connection portion which includes a cable extending from the storage to the electrode array and provided with a leading wire electrically connecting the electrode array to the electronic circuit unit, and a fluidic channel through which fluid flows between the storage and the electrode array.

### CROSS-REFERENCE TO RELATED APPLICATION

This application claims priority to and the benefit of Korean Patent Application No. 10-2017-0029646, filed on Mar. 8, 2017, the disclosure of which is incorporated herein by reference in its entirety.

### BACKGROUND OF THE INVENTION

### Field of the Invention

Hereinafter, embodiments relate to a neural electrode device comprising a neural electrode array for measuring neural signals or giving electrical stimulus to nerves and a micro fluidic channel for delivering drug.

### Description of the Related Art

3-dimensional invasive type neural electrode arrays have been developed which measure neural signals from peripheral nerves of a subject or neurons of a brain or provide stimulus to the peripheral nerves or neurons to study or cure a disease of the subject. Generally, since a 3-dimensional invasive type neural electrode array based on silicon or glass is made of a hard and fragile material, thus it is difficult to couple micro fluidic channel. In addition, in the case of a 3-dimensional invasive type neural electrode array based on a flexible supporter, there is no case of trying to couple fluidic channel to mount a function of delivering drug. For example, Korean Patent Registration No. 10-1118349 discloses a transplantable neural stimulus electrode having a fluid storage unit.

### SUMMARY OF THE INVENTION

An object according to an embodiment is to provide a neural electrode device in which a neural electrode array and a connection portion comprising a micro fluidic channel capable of delivering fluid such as drug are integrated.

Another object according to an embodiment is to provide a neural electrode device with biocompatibility.

Still another object according to an embodiment is to provide a neural electrode device which continuously supplies fluid to a subject in the long term.

Still another object according to an embodiment is to precisely adjust the amount of fluid supplied from a storage storing fluid to a fluidic channel.

Still another object according to an embodiment is to provide a neural electrode device which stably reads bio-signals from a subject in the long term.

A neural electrode device according to an embodiment may comprise: a storage in which fluid is stored; an electrode array which delivers the fluid to a subject, acquires bio-signals from the subject, or provides stimulus to the subject; an electronic circuit unit which allows the fluid to flow from the storage to the electrode array, processes the bio-signals acquired from the electrode array, or provides stimulus signals to the electrode array; and a connection portion which includes a cable extending from the storage to the electrode array and provided with a leading wire electrically connecting the electrode array to the electronic circuit unit, and a fluidic channel through which fluid flows between the storage and the electrode array.

The connection portion may further comprise a plurality of columns installed in the fluidic channel.

The plurality of columns may be disposed in zigzag in the fluidic channel.

A cross-sectional shape of the fluidic channel may be an arched shape.

The neural electrode device may further comprise a valve which is installed between the storage and the fluidic channel, and adjusts the amount of fluid supplied from the storage to the fluidic channel.

The connection portion may be formed of a flexible polymer material such that the connection portion is curved from the outside of the subject to the inside of the subject when the storage is disposed inside or outside the subject and the electrode array is inserted into the subject and comes in contact with neural tissues.

The electrode array may comprise: a substrate above which the fluidic channel passes; a plurality of probes which are installed on the substrate and are inserted to the subject; and a fluid outlet which is formed on the substrate, is connected to the fluidic channel, and performs fluid communication with the fluidic channel.

The fluid outlet may be formed in at least one probe of the plurality of probes.

The fluid outlet may extend from the substrate to the subject and may have a tubular shape.

The plurality of probes may be disposed on the substrate in a direction vertical to an extension direction of the cable.

A neural electrode device according to an embodiment may comprise: a storage in which fluid is stored; a connection portion which includes a cable extending from the storage in a length direction, is formed of a flexible material, and curves in a plurality of directions, a fluidic channel through which the fluid flows, and a plurality of columns installed in the fluidic channel; an electrode array which includes a substrate installed at the end of the fluidic channel passing above the substrate, a plurality of probes disposed on the substrate in a direction vertical to an extension direction of the connection portion, and a fluid outlet connected to the fluidic channel and delivering the fluid to a subject; and an electrode circuit unit which allows the fluid to flow from the storage to the electrode array, processes bio-signals acquired from the electrode array, or provides stimulus signals to the electrode array.

In the neural electrode device according to the embodiment, the connection portion and the electrode array are integrated and used, and there is space efficiency when being used for the subject.

The neural electrode device according to the embodiment has biocompatibility.

The neural electrode device according to the embodiment can continuously supply fluid to a subject in the long term.

The fluidic channel in the connection portion of the neural electrode device according to the embodiment can supply fluid to a subject even in folding and twisting.

The neural electrode device according to the embodiment can stably read bio-signals from a subject in the long term.

The advantages of the neural electrode device according to the embodiment are not limited to what are described above, and the other advantages which are not described can be clearly understood for persons skilled in the art from the following description.

While the present invention has been described with respect to the specific embodiments, it will be apparent to those skilled in the art that various changes and modifications may be made without departing from the spirit and scope of the invention as defined in the following claims.

### BRIEF DESCRIPTION OF THE DRAWINGS

FIG. 1 is a schematic diagram illustrating a neural electrode device according to an embodiment;

FIG. 1A is a schematic diagram illustrating a neural electrode device according to an embodiment;

FIG. 2 is a schematic perspective view illustrating a neural electrode device according to an embodiment;

FIG. 3 is a schematic cross-sectional view illustrating a cross section (A-A′) of a connection portion according to an embodiment;

FIG. 4 is a schematic cross-sectional view illustrating a cross section of an electrode array and a fluid outlet according to an embodiment;

FIG. 5 is a diagram illustrating a method for forming a fluid outlet in the vicinity of an electrode array according to an embodiment;

FIG. 6 is a schematic cross-sectional view illustrating a cross section of an electrode array and a fluid outlet according to an embodiment;

FIG. 7 is a diagram illustrating a method for forming a fluid outlet in the vicinity of an electrode array according to an embodiment;

FIG. 8 is a schematic cross-sectional view illustrating a cross section of an electrode array and a fluid outlet according to an embodiment;

FIG. 9 is diagram illustrating a method for forming a fluid outlet in the vicinity of an electrode array according to an embodiment;

FIG. 10 is a schematic cross-sectional view illustrating a cross section of an electrode array and a fluid outlet according to an embodiment;

FIG. 11 is a diagram illustrating a method for removing a part of probes of an electrode array and forming a fluid outlet according to an embodiment;

FIG. 12 is a schematic partial cross-sectional perspective view illustrating a part of a fluidic channel of a connection portion of a neural electrode device according to an embodiment;

FIG. 13 is a schematic cross-sectional view illustrating a cross section of a connection portion according to an embodiment; and

FIG. 14A to FIG. 14G are diagrams illustrating a method for manufacturing a connection portion according to an embodiment.

### DETAILED DESCRIPTION OF THE PREFERRED EMBODIMENTS

Hereinafter, embodiments will be described in detail with reference to exemplary drawings. In assigning reference numerals and signs to constituent elements of each drawing, it should be noted to have the same reference numerals and signs to the same constituent elements although they are illustrated on different drawings. In addition, in describing embodiments, when it is determined that specific description for the related known configurations or functions interferes with understanding embodiments, the detailed description is omitted.

In addition, in describing constituent elements of embodiments, the terms such as “first, second, A, B, (a), and (b)” may be used. Such terms are only to distinguish the constituent elements from the other constituent elements, and the nature, sequence, order, or the like of the constituent elements is not limited by the terms. When it is described that any constituent element is “connected” or “coupled” to another constituent element, it should be understood that the constituent element is directly connected or coupled to another constituent element, but still another constituent element may be “connected” or “coupled” between the constituent elements.

A constituent element included in any one embodiment and a constituent element including a common function will be described by using the same term in other embodiments. Unless otherwise stated, the explanation described in any one embodiment may be applied to other embodiments, and specific description thereof is omitted in an overlapped range.

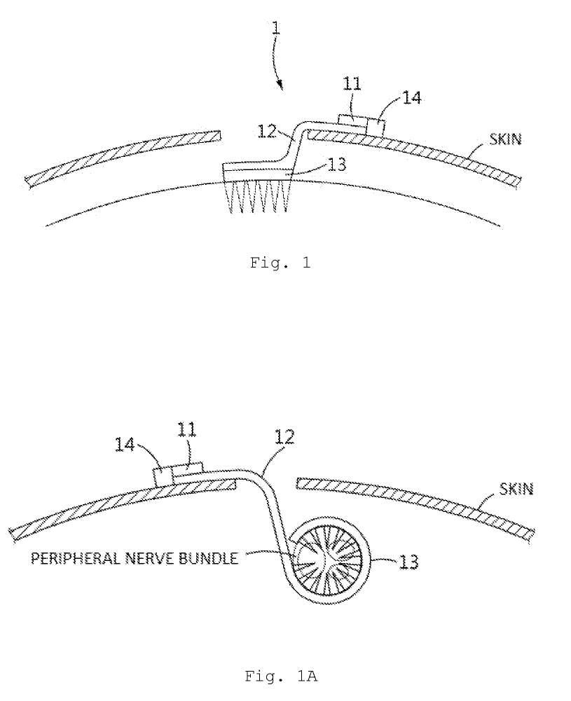

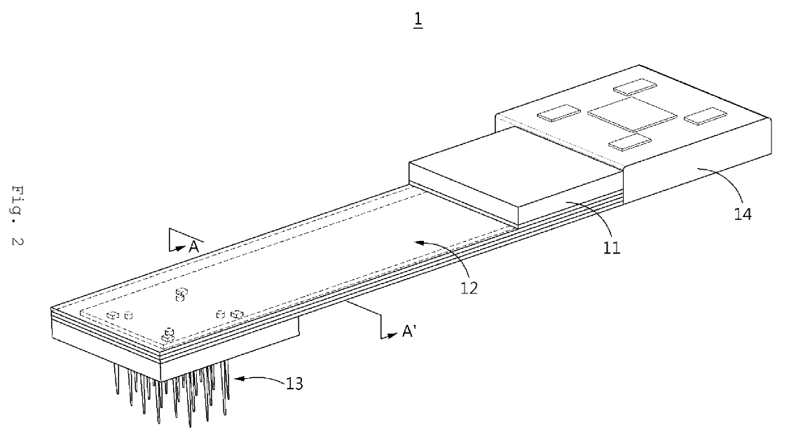

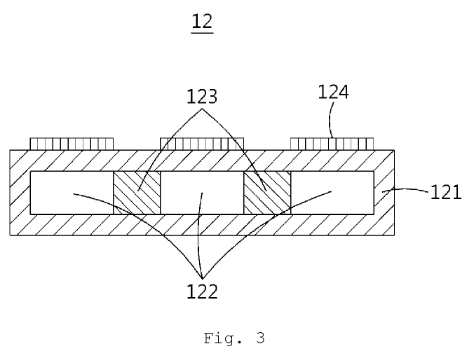

FIG. 1 is a schematic diagram illustrating a neural electrode device according to an embodiment, FIG. 1A is a schematic diagram illustrating a neural electrode device according to an embodiment, FIG. 2 is a schematic perspective view illustrating a neural electrode device according to an embodiment, and FIG. 3 is a schematic cross-sectional view illustrating a cross section of a connection portion according to an embodiment.

Referring to FIG. 1, FIG. 1A, FIG. 2, and FIG. 3, a neural electrode device 1 according to an embodiment may comprise a storage 11, a connection portion 12, an electrode array 13, an electronic circuit unit 14, and a valve (not illustrated). The neural electrode device 1, one end of which comes in contact with a subject and the other end is disposed at a position separated as much as a length between the subject and the connection portion 12, may deliver fluid to the subject, detect bio-signals from the subject, or provide electrical stimulus to the subject. Herein, the subject may be a human body, an animal, or the like, and the neural electrode device 1 may be connected to neural tissues of the human body, the animal, or the like. Herein, the fluid generally means drug, but is not limited thereto. FIG. 1 illustrates that one end of the neural electrode device 1 is disposed on the outer surface of a brain, the other end thereof is disposed on the outer surface of a skull, probes acquire bio-signals from neurons or stimulate neurons, and fluid is delivered to a brain, but is not limited thereto. In addition, FIG. 1A illustrates that one end of the neural electrode device 1 is disposed on the outer surface of peripheral nerves, the other end thereof is disposed around the peripheral nerves, probes acquire signals from neurons in peripheral nerves or stimulate neurons, and fluid is delivered to peripheral nerves, but is not limited thereto. For example, one end of the neural electrode device 1 may be disposed on the outer surface of skin, not only outer surface of a skull, of a subject to deliver fluid transdermally into the subject.

The storage 11 may retain and store fluid therein. In other words, fluid may be stored in the storage 11. However, the present invention is not limited thereto, and the storage 11 may receive fluid from the outside.

The connection portion 12 may deliver the fluid from the storage to the electrode array 13 and deliver bio-signals from the electrode array 13 to the electronic circuit unit 14. The connection portion 12 may include a cable 121, a fluidic channel 122, a column 123, and a leading wire 124.

The cable 121 may connect the storage 11 to the electrode array 13, and may connect the electrode array 13 to the electronic circuit unit 14. The electronic circuit unit 14 may be disposed at one end of the cable 121, the storage 11 may be disposed at a part of one surface of the cable 121, and the electrode array 13 may be disposed at a part of the other surface of the cable 121. Specifically, when the storage 11 is disposed on the upper surface or the lower surface of the cable 121, the electrode array 13 may be disposed on the lower surface or the upper surface of the cable 121, and the storage 11 and the electrode array 13 may be disposed at both ends of the cable 121 to be separated. The cable 121 may extend from the storage 11 to the electrode array 13 in a length direction. In other words, the storage 11, the electrode array 13, and the cable 121 may be formed integrally.

According to such a structure, in allowing fluid to flow from the storage 11 to the electrode array 13, it is easier to control the fluid flowing from the storage 11 to the electrode array 13 and it is possible to continuously inject fluid to a subject in the long term when the storage 11 and the electrode array 13 are formed integrally than when they are separated by a considerable distance.

The cable 121 may be formed of a flexible material. For example, the cable 121 may be formed of a polymer material. The polymer material may be polyethylene terephthalate (PET), polyethylene naphthalate (PEN), polyimide (PI), polymethyl methacrylate (PMMA), parylene, polydimethylsiloxane (PDMS), SU-8, or the like.

In this case, the storage 11 disposed on one surface of the cable 121 and the electrode array 13 disposed on the other surface of the cable 121 may be positioned at portions different from each other when the cable 121 is curved in a plurality of directions. In other words, when the storage 11 is disposed outside the subject and the electrode array 13 is inserted into the subject, the cable 121 may be curved from the outside of the subject to the inside of the subject.

According to such a structure, even without inserting the storage 11 into the subject, nerves can be stimulated and only the electrode array 13 injecting fluid can be inserted into the subject.

The fluidic channel 122 is a space through which fluid flows between the storage 11 and the electrode array 13, and may be formed to perform fluid communication with the storage 11 and the electrode array 13, respectively. The fluidic channel 122 may be formed as a path through which fluid flows in the cable 121. For example, the fluidic channel 122 may be formed such that one surface and the other surface of the cable 121 are parallel with each other.

The column 123 may control a speed of fluid flowing through the fluidic channel 122. The column 123 may be formed to extend from one surface of the cable 121 to the other surface of the cable 121, and the fluid does not pass through the portion with the column 123, and the fluid may flow only through a space provided with the fluidic channel 122. A plurality of columns 123 may be installed in the cable 121 to be separated from each other. In this case, the columns 123 may be installed in the cable 121 as a separate individual configuration, but may be formed to be integrated with the cable 121.

According to such a structure, even when the cable 121 is curved or twisted in any direction, it is possible to prevent the fluidic channel 122 from being clogged by the columns 123, and thus it is possible to continuously supply the fluid from the storage 11 to the subject in the long term.

The leading wire 124 may deliver bio-signals acquired from the electrode array 13 to the electronic circuit unit 14, or may deliver stimulus signals from the electronic circuit unit 14 to the electrode array 13. The leading wire 124 may electrically connect the electrode array 13 to the electronic circuit unit 14.

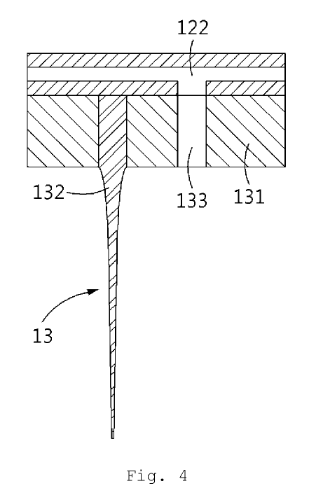

The electrode array 13 may stimulate neurons of the subject, may inject fluid to the subject, or may acquire bio-signals from the neurons of the subject. A specific configuration of the electrode array 13 and a specific configuration of the electrode array 13 as an additional embodiment will be described later with reference to FIG. 4 to FIG. 11.

The electronic circuit unit 14 may allow the fluid in the storage 11 to flow from the storage 11 to the electrode array 13, may receive the bio-signals acquired from the electrode array 13 and process the bio-signals, or may provide stimulus signals to the electrode array 13. In this case, the electronic circuit unit 14 may amplify the delivered bio-signals or may transmit the bio-signals to an external recording device.

The valve (not illustrated) may adjust the amount of fluid supplied from the storage 11 to the fluidic channel 122 of the connection portion 12. The valve may be installed between the storage 11 and the fluidic channel 122. According to such a structure, the amount of fluid supplied from the storage 11 to the fluidic channel 122 is precisely adjusted, thereby controlling the amount of fluid delivered depending on the situation.

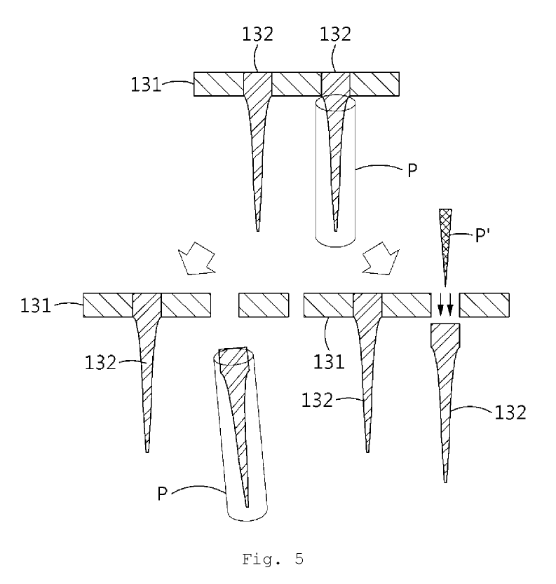

FIG. 4 is a schematic cross-sectional view illustrating a cross section of an electrode array and a fluid outlet according to an embodiment, and FIG. 5 is a diagram illustrating a method for forming a fluid outlet in the vicinity of an electrode array.

Referring to FIG. 4, an electrode array 13 according to an embodiment may comprise a substrate 131, a plurality of probes 132, and a fluid outlet 133.

The substrate 131 may be installed at a part of one surface of the cable 121 (see FIG. 2 and FIG. 3). In this case, the fluidic channel 122 (see FIG. 3) formed in the cable 121 may pass above the substrate 131. In addition, the substrate 131 may be formed of a flexible material to be disposed according to a structure (e.g., curved surface) of a subject. For example, the substrate 131 may be formed of an elastomer.

The plurality of probes 132 may be inserted into a subject to stimulate nerves of the subject or read bio-signals from the nerves of the subject. The plurality of probes 132 may penetrate the substrate 131 and one end of which may be installed in the substrate 131.

The fluid outlet 133 may perform fluid communication with the fluidic channel 122. To this end, the fluid outlet 133 may be formed on the substrate 131, and may be connected to the fluidic channel 122 passing above the substrate 131. Specifically, the fluid outlet 133 may be coupled at a position where the plurality of probes are installed on the substrate 131 patterned to position the plurality of probes 132.

According to such a structure, when the plurality of probes 132 are inserted into the subject, fluid can be directly delivered to the subject through the fluid outlet 133.

Referring to FIG.5, when a position where a plurality of probes 132 is installed on the substrate 131 is patterned on the substrate 131 and the plurality of probes 132 are installed on the substrate 131, a probe 132 at a desired position may be removed by using a micro-punch P or a tweezer P′. A fluid outlet may be formed with the formed hole.

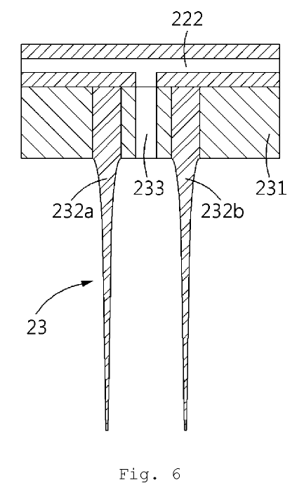

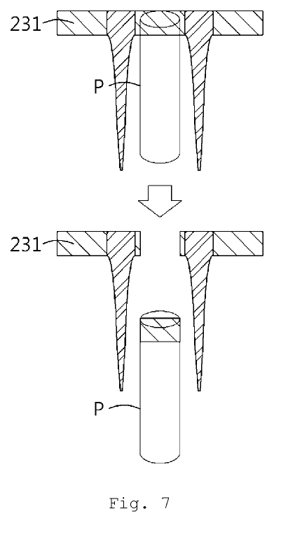

FIG. 6 is a schematic cross-sectional view illustrating a cross section of an electrode array and a fluid outlet according to an embodiment, and FIG. 7 is a diagram illustrating a method for forming a fluid outlet in the vicinity of an electrode array according to an embodiment.

Referring to FIG. 6, an electrode array 23 according to an embodiment may comprise a substrate 231, a plurality of probes 232a and 232b, and a fluid outlet 233 connected to a fluidic channel 222. The fluid outlet 233 may be formed on the substrate 231 between the plurality of probes 232a and 232b.

Referring to FIG. 7, when the plurality of probes 232a and 232b are installed on the substrate 231, a hole may be generated at a part of the substrate 231 between the plurality of probes 232a and 232b by using a micro-punch P without removing a probe at a desired portion. A fluid outlet may be formed with the formed hole.

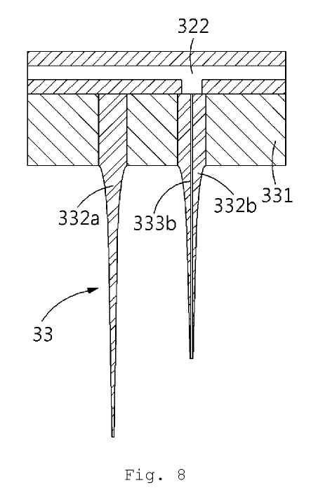

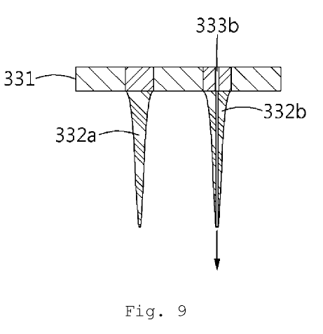

FIG. 8 is a schematic cross-sectional view illustrating a cross section of an electrode array and a fluid outlet according to an embodiment, and FIG. 9 is a diagram illustrating a method for forming a fluid outlet in the vicinity of an electrode array according to an embodiment.

Referring to FIG. 8, an electrode array 33 according to an embodiment may comprise a substrate 331, a plurality of probes 332a and 332b, and a fluid outlet 333b connected to a fluidic channel 322. The fluid outlet 333b may be formed in at least one probe 332b of the plurality of probes 332a and 332b. According to such a structure, when the plurality of probes 332a and 332b are inserted into a subject, it is possible to directly deliver fluid to a stimulated portion while stimulating nerves. According to the FIG. 8, the plurality of probes 332a and 332b have different length, but the length of the plurality of probes 332a and 332b may be identical or be different.

Referring to FIG. 9, when the plurality of probes 332a and 332b are installed on the substrate 331, a fluid outlet 333b may be formed in the probe 332b at a desired portion by irradiating the inside of the probe 332b of the desired portion on one surface of the substrate 331 with laser or performing deep reactive-ion etching. Alternatively, a probe including a fluid outlet therein may be made by pulling a tubular glass tube while applying heat thereto.

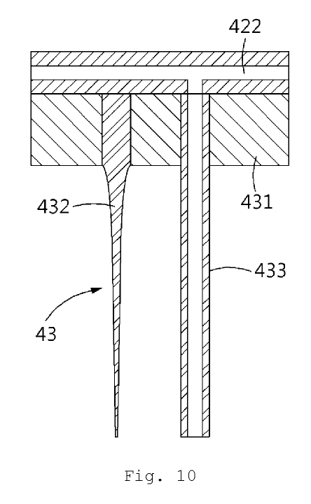

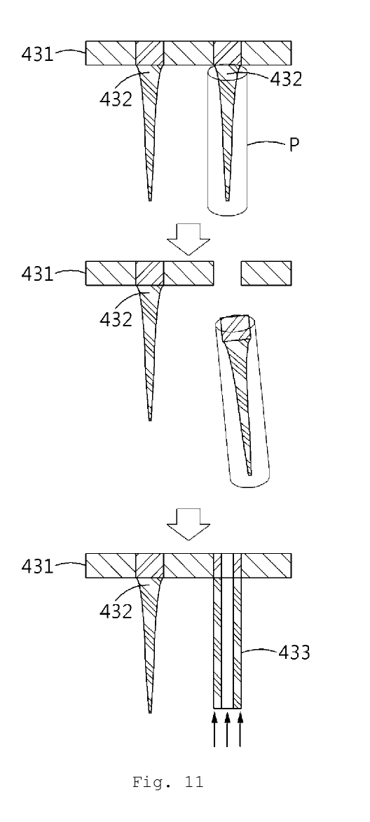

FIG. 10 is a schematic cross-sectional view illustrating a cross section of an electrode array and a fluid outlet according to an embodiment, and FIG. 11 is a diagram illustrating a method for removing a part of probes of an electrode array and forming a fluid outlet according to an embodiment.

Referring to FIG. 10, an electrode array 43 according an embodiment may comprise a substrate 431, a plurality of probes 432, and a fluid outlet 433 connected to a fluidic channel 422. The fluid outlet 433 may extend from one surface of the substrate 431 and may have a tubular shape. According to such a structure, when the probes 432 are inserted into a subject, it is possible to deliver a larger amount of fluid to the subject.

Referring to FIG. 11, when the plurality of probes 432 are installed on the substrate 431, a part of probes 432 of the plurality of probes 432 may be removed by using a micro-punch P. A tubular fluid outlet 433 may be installed at the removed position on the substrate 431.

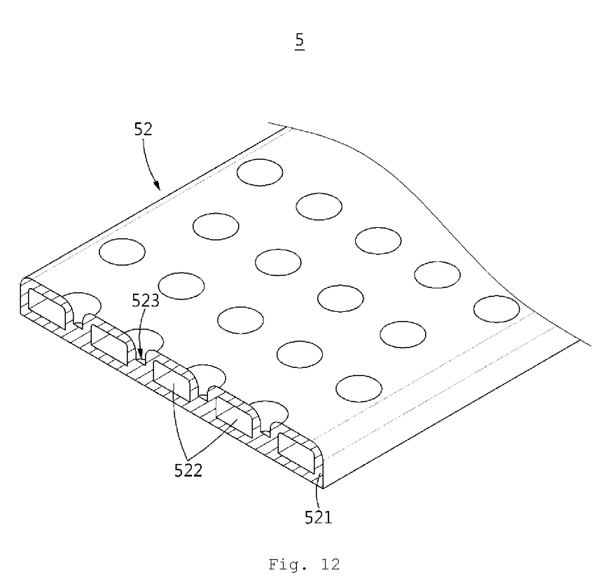

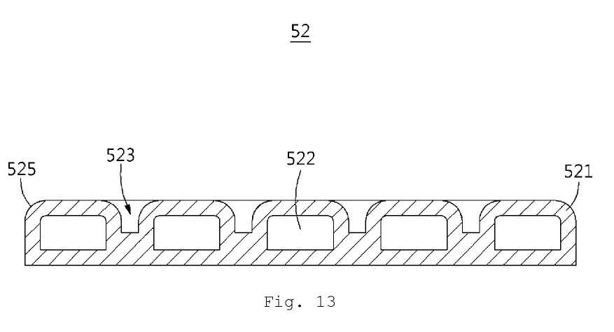

FIG. 12 is a schematic partial cross-sectional perspective view illustrating a part of a fluidic channel of a connection portion of a neural electrode device according to an embodiment, and FIG. 13 is a schematic cross-sectional view illustrating a cross section of a connection portion according to an embodiment.

Referring to FIG. 12 and FIG. 13, a neural electrode device 5 according to an embodiment may comprise a storage (not illustrated), a connection portion 52, an electrode array (not illustrated), an electronic circuit unit (not illustrated), and a valve (not illustrated).

The connection portion 52 may include a cable 521, fluidic channel 522, and columns 523.

The fluidic channel 522 may have an arched cross section. Specifically, a part of a corner of the fluidic channel 522 may be rounded to form a curved portion 525.

The columns 523 may be disposed in the cable 521 to be separated from each other. In this case, the column 523 may be positioned between the plurality of fluidic channel 522.

According to such a structure, even when the cable 521 is curved or twisted in any direction, it is possible to prevent the fluidic channel 522 from being clogged by the column 523. In other words, the neural electrode device 5 can continuously deliver fluid to a subject in the long term.

The columns 523 may be disposed in zigzag in the fluidic channel 522. When a first row forming at least one column and a second row forming at least one column are disposed to be in parallel and adjacent to each other, the column of the first row and the column of the second row may not be positioned in parallel in a length direction of the cable 521.

According to such a structure, flow F of the fluid flowing in the fluidic channel 522 does not flow in the length direction of the cable 521 and turns on the column 523. Accordingly, a speed of the fluid may decrease, and it is possible to control the flow F of the fluid when the fluid is delivered from the storage in which drug is stored to the subject through the electrode array.

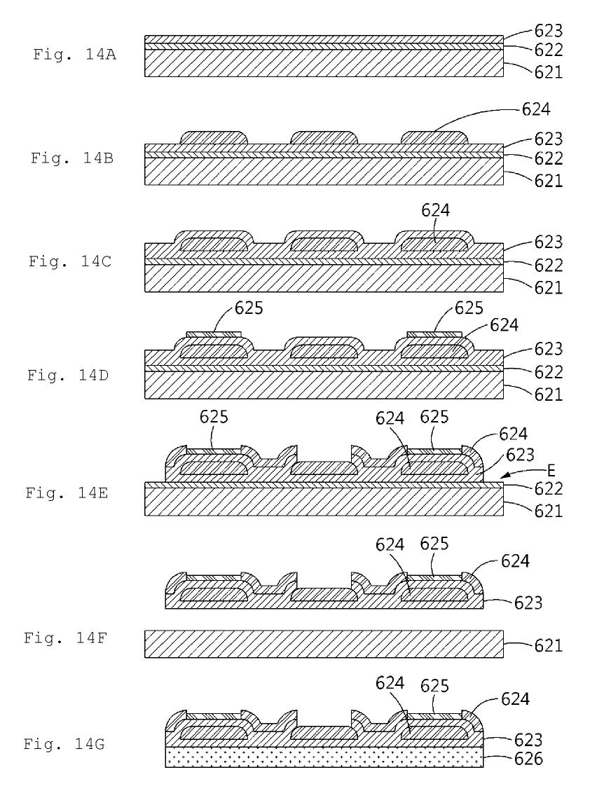

FIG. 14A to FIG. 14G are diagrams illustrating a method for manufacturing a connection portion according to an embodiment.

Referring to FIG. 14A to FIG. 14G, FIG. 14A illustrates a substrate preparation process, in which a sacrificial layer 622 is formed on a carrier wafer 621 and parylene 623 is deposited thereon.

FIG. 14B illustrates a fluidic channel patterning process, in which a photoresist 624 is patterned on the deposited parylene 623.

FIG. 14C illustrates a base substrate process, in which the parylene 623 is deposited on the photoresist 624 to surround the photoresist 624.

FIG. 14D illustrates a conductive line patterning process, in which metal 625 as a conductive material is patterned on the deposited parylene 623 at the position where the photoresist 624 is deposited.

FIG. 14E illustrates an electrode active area process for forming an inlet and an outlet of a fluidic channel, in which the parylene 623 deposited at the positions of both ends of the carrier wafer 621 is removed €, and the photoresist 624 is applied onto the parylene 623 deposited around the metal 625.

FIG. 14F illustrates a fluidic channel forming process, in which every sacrificial layer 622 between the carrier wafer 621 and the parylene 623 is removed.

FIG. 14G illustrates a support structure process, in which the carrier wafer 621 is removed, and a polyimide cable 626 is attached to the lower portion of the deposited parylene 623.

As described above, the embodiments have been described by defined embodiments and drawings, but persons skilled in the art can variously changed and modified from the description. For example, even when the described techniques are performed in order different from the described method, and/or the constituent elements such as the described system, structure, device, and circuit are coupled or combined in a form different from the described method, or are replaced or substituted by other constituent elements or equivalents, it is possible to achieve a proper result.

1. A neural electrode device comprising:
a storage for storing fluid;
an electrode array for delivering fluid to a subject, acquiring bio-signals from the subject, or providing stimulus to the subject;
an electronic circuit unit for allowing fluid to flow from the storage to the electrode array, processing bio-signals acquired from the electrode array, or providing stimulus signals to the electrode array; and
a connection portion which includes a cable extending from the storage to the electrode array and provided with a leading wire electrically connecting the electrode array to the electronic circuit unit, and a fluid channel for fluid flow between the storage and the electrode array.

2. The neural electrode device according to claim 1, wherein the connection portion comprises:
a plurality of columns installed in the fluid channel.

3. The neural electrode device according to claim 2, wherein the plurality of columns are disposed in a zigzag in the fluid channel.

4. The neural electrode device according to claim 3, wherein a cross-sectional shape of the fluid channel is arched shape.

5. The neural electrode device according to claim 1, comprising:
a valve which is installed between the storage and the fluid channel, for adjusting an amount of fluid supplied from the storage to the fluid channel.

6. The neural electrode device according to claim 1, wherein the connection portion is formed of a flexible polymer material such that the connection portion is curved from an outside of the subject to an inside of the subject when the storage is disposed inside or outside the subject and the electrode array is inserted into the subject and comes in contact with neural tissues.

7. The neural electrode device according to claim 1, wherein the electrode array comprises:
a substrate above which the fluid channel passes;
a plurality of probes which are installed on the substrate and are configured to be inserted to a subject; and
a fluid outlet configured to be formed on a substrate, connected to the fluid channel, and to provide fluid communication with the fluid channel.

8. The neural electrode device according to claim 7, wherein the fluid outlet is formed in at least one probe of the plurality of probes.

9. The neural electrode device according to claim 7, wherein the fluid channel is arranged to extend from the substrate to the subject and has a tubular shape.

10. The neural electrode device according to claim 7, wherein the plurality of probes are disposed on a substrate in a direction vertical to an extension direction of the cable.

11. A neural electrode device comprising:
a storage for storing fluid;
a connection portion which includes a cable extending from the storage in a length direction, is formed of a flexible material, and is curved in a plurality of directions, a fluid channel for fluid flow, and a plurality of columns installed in the fluid channel;
an electrode array which includes a substrate installed at the end of the fluid channel passing above the substrate, a plurality of probes disposed on the substrate in a direction vertical to an extension direction of the connection portion, and a fluid outlet connected to the fluid channel for delivering fluid to a subject; and
an electrode circuit unit for allowing fluid to flow from the storage to the electrode array, processing bio-signals acquired from the electrode array, or providing stimulus signals to the electrode array.
